(19) United States
(12) Patent Application Publication
(10) Pub. No.: US 2003/0130587 A1
(43) Pub. Date: Jul. 10, 2003

(54) Non-invasive method of cardiac output measurement through assessment of skin thermal response

(76) Inventor: Shahin Ahmedov, Lefkosha (TR)

Correspondence Address: Shahin Ahmedov, P.O. Box 670, Near East University, Lefkosha, K.K.T.C/Mersin, 10, TR

(21) Appl. No.: 09/988,961

(22) Filed: Jan. 7, 2002

(51) Int. Cl. A61B 5/00; A61B 5/02

(52) U.S. Cl. 600/526; 600/504; 600/549

(57) ABSTRACT

The warming ability of skin has been measured by thermometer, which sensor was cooled upto 17-18 deg.C. before its contact with skin.

The warming rate of thermometer during its 30 sec contact with forearm skin depended on position of the sensor of that thermometer: the warming was faster over pulsation of a.radialis in compare to its position on the back of the wrist. Besides, the warming rate on former position (i.e. over-artery position) well-correlated with the body surface area (r=0.65) and pulse pressure (r=0.58), whereas there were no any correlations of results obtained from latter position (i.e. back of the wrist).

The bioheat transfer, as the reason of above-mentioned warming over-artery position, was provided by blood flow within a.radialis, i.e. forced convection. Thus, through asessment of warming rate of cooled thermometer over a.radialis pulsation and application of the laws of thermodynamics we can measure cardiac output of the human body.

### PROBLEM

There are different measurable parameters in medicine, characterizing cardiovascular system: blood pressure, pulse rate, cardiac output, stroke volume etc. Cardiac Output (CO) is considered the most important among them. It presents total amount of blood in liters (L) pumpted out by heart through circulation in 1 min. Under normal conditions CO=4-8 L/min and its amount depends on Body Surface Area (BSA, m2): the the larger BSA so the more CO should be. Besides CO is equal to Pulse Rate multiplied by the Stroke Volume, SV, the amount of blood in liters pumpted out by heart through circulation in one contraction. Usually SV is in close relation with the Pulse Pressure, PP, the difference between Systolic and Diastolic Blood pressures.

All existing techniques providing measurement of CO either precise but potentially risky for health or safe but expensive and imprecise. The method offered in this record is safe, non-expensive and non-invasive (i.e. through skin, without blood contact) way of CO measurement through assessment of the thermal response of the skin over artery by digital thermometer (FIG. 1). This approach seems to be especially important today, when preventive health technology becomes a key factor of the health improvement through the world.

### INVENTED PROCESS

Experimentally if we could place a small cold solid object directly on the wall of large blood vessel it would be warmed after sometime. And the rate of its warming will depend on (i) temperature gradient between blood vessel and that object, (ii) size of that object and (iii) RATE OF THE BLOOD FLOW. Thus, through assessment of these thermal changes under steady-state conditions (i.e. constant inner temperature and size of the object) we can measure the rate of the blood flow and consequently the cardiac output.

Abovementioned experiment is impossible to be carry out in clinical situation. However, there are some parts of the human body, where artery passes so close to the body surface, that we can easy palpate arterial pulsation there (e.g. arterial pulsation on the front of the wrist) and we can expect different thermal body response on cold challenge over there in compare to other parts of the body. Aiming to combine COLD STIMULI OF THE SKIN OVER ARTERY AND THE MEASURE OF THAT SKIN RESPONSE ON IT I have used THERMOMETER itself as COLD STIMULI (FIG. 1). Statistical evaluation of data, obtained in 26 volunteers allows to consider abovementioned thermal response of the skin over artery on exposure of cold thermometer as the result of blood flow rate within that vessel.

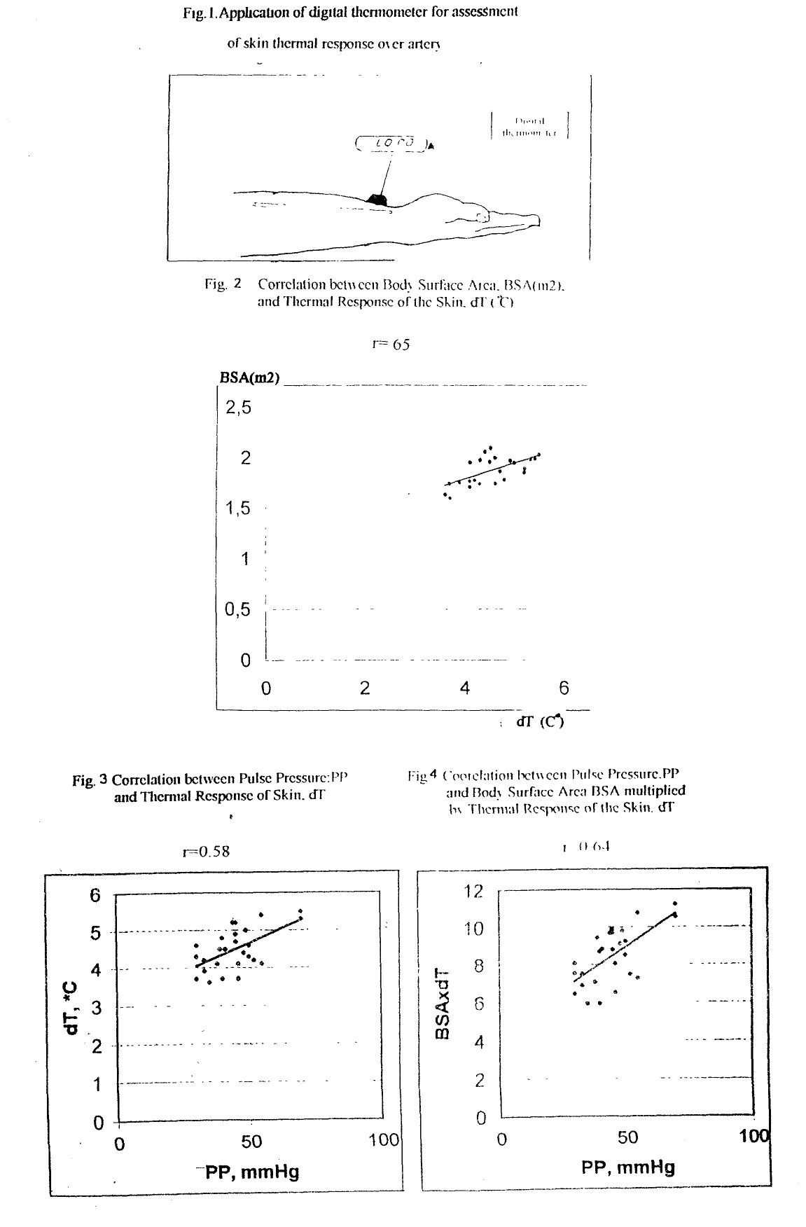

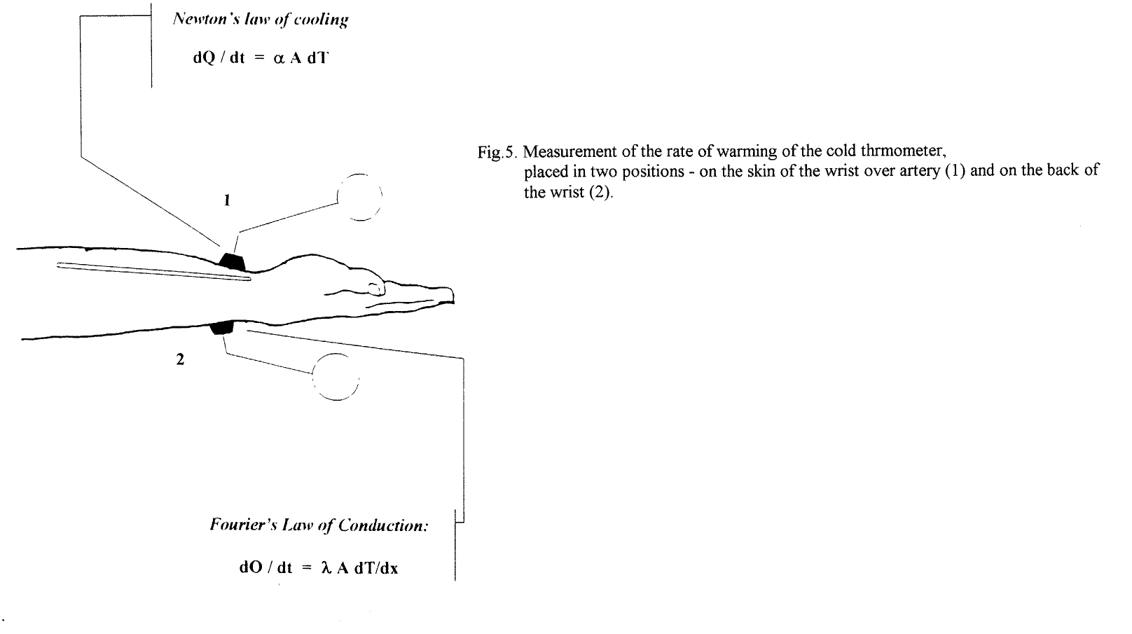

This thermal responsse of the skin (dT) has been in significant correlation with BSA (r=0.65, FIG. 2) and PP (r=0.58, FIG. 3). The latter correlation increased upto 0.64 when dT was multiplied by BSA (FIG. 4). Proper application of well-known laws of thermodynamics with their corresponding formulas for the heat transfer with forced convection can allow to calculate the rate of the blood flow and consequently cardiac output. Similarly, simultaneous measurement of CO and dT in the group of patients can give the parameters of dT which couple with the corresponding data of CO.

### SPECIFICATION

The warming of previously cooled digital thermometer during its exposure on the wrist is the matter of heat transfer of the body. However the mechanism of this bioheat transfer will depennd on the position of the above-mentioned thermometer on the wrist. If we use the position just over the a.radialis, then the mechanism of heat transfer, which is the only reason of warming of the previously cooled digital thermometer, is FORCED CONVECTION. The latter is calculated by the usage of Newton's Law of Cooling:

dQ/dt=α·A·dT (1)

where dQ/dT is the rate of heat flow, A is the surface area of the sensor of the thermometer, dT is the temperature gradient between wrist and cooled thermometer, and a is the heat transfer coefficient.

If we use the position on the dorsal side of the wrist, then the mechanism of heat transfer, leading to the warming of cold thermometer, is CONDUCTION, which is the matter of Fourier's Law of Conduction:

dQ/dt=λ·A˜dT/dx (2)

where dx is the distance between a.radialis and the sensor and the λ is thermal

Taking into consideration the fact that under steady-state conditions the overall heat flow of the wrist is the same it is reasonable to join equations (1) and (2) to get heat transfer coefficient equation.

α·A·dT=λ·A·dT/dx (3)

α=λ/dx (4)

Then by usage of the Nusselt number, Prandtl number and Reynolds number (includes the parameter of velocity) the blood flow velocity within a.radialis can be calculated and cardiac output can be approximated from obtained result.

CLAIM: CARDIAC OUTPUT CAN BE MEASURED NON-INVASIVLY THROUGH THE MEASUREMENT OF WARMING ABILITY OF THE SKIN ON THE WRIST BY THE PREVIOUSLY COOLED ONE-FLAT-SURFACE SENSOR OF THE DIGITAL THERMOMETER AND CONSEQUTIVE APPLICATION OF LAWS OF HEAT TRANSFER, DESCRIBED IN SPECIFICATION.

[image]

1. CARDIAC OUTPUT CAN ME MEASURED NON-INVASIVLY THROUGH THE MEASUREMENT OF WARMING ABILITY OF THE SKIN ON THE WRIST BY PREVIOUSLY COOLED ONE-FLAT-SURFACE SENSOR OF THE DIGITAL THERMOMETER AND CONSEQUTIVE APPLICATION OF LAWS OF HEAT TRANSFER, DESCRIBED IN SPECIFICATION:
